SEQUENCE LISTING (2 sequences)

SEQ ID NO: 1 - DNA, 6710 bp, Artificial
  Designed plasmid named pCTx
gacggatcgggagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatccactagtccagtgtggtggaattctgcagatatccagcacagtggcggccgctcgagtctagacatatggtttaaactgatcatgagggattctctgctccgtcagccacactcctagggcattgtgatgcttacataagcaagtggctcaaatgattcagtattctgtggtccctggggctttattcggagactctgtcggggtgagctgacaatttagactcaattctttctgcccacctggccatccaggaagtgttccgacggttggcacgcagccagggctggcacaggaacgtgggttccaaacaacgctcgtatggactgaaaagatacgcaggcgtcaaatatgcaaacgagatagattatttaaggagactgtgtgtgactgaaaagcaacattaaaaaataataaatttcattttctacatgaaaaaaaaaaaaaaaaaaaaaaaaaaggaaaaaaaaaaaggccggagcggccgccaccgcggtggagctccagcttttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcactgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcagtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 6375 bp, Artificial
  Designed plasmid named pCTIx
gacggatcgggagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatccactagtccagtgtggtggaattctgcagatatccagcacagtggcggccgctcgagtctagacatatggtttaaactgatcatgagggattctctgctccgtcagccacactcctagggcattgtgatgcttacataagcaagtggctcaaatgattcagtattctgtggtccctggggctttattcggagactctgtcggggtgagctgacaatttagactcaattctttctgcccacctggccatccaggaagtgttccgacggttggcacgcagccagggctggcacaggaacgtgggttccaaacaacgctcgtatggactgaaaagatacgcaggcgtcaaatatgcaaacgagatagattatttaaggagactgtgtgtgactgaaaagcaacattaaaaaataataaatttcattttctacatgaaaaaaaaaaaaaaaaaaaaaaaaaaggaaaaaaaaaaaggccggagcggccgccaccgcggtggagctccagcttttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgcccgggagcttgtatatccattttcggatctgatcagcactgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgcatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcagtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2005/0064547 A1
(43) Pub. Date: Mar. 24, 2005

(54) Vectors and transfected cells

(76) Inventors: Arthur M. Brown, Brecksville, OH (US); Barbara A. Wible, Cleveland, OH (US)

Correspondence Address: FLESHNER & KIM, LLP, P.O. Box 221200, Chantilly, VA, 20153-1200, US

(21) Appl. No.: 10/668,496

(22) Filed: Sep. 24, 2003

(51) Int. Cl. C12Q 1/68; C07H 21/04; C12N 9/64; C07K 14/705; C12N 15/85; C12N 5/06

(52) U.S. Cl. 435/69.1; 435/455; 435/320.1; 435/353; 530/350; 435/226; 536/23.5

(57) ABSTRACT

Disclosed are vectors for cloning and expressing nucleic acid sequences, methods of transfecting cells with these vectors, transfected cells containing these vectors, and antibiotic resistance cassettes. For instance, the vector may include, from upstream to downstream, a first promoter, at least one cloning site, a rat Kv2.1 polyadenylation sequence, and an origin of replication. As another example, the vector includes, from upstream to downstream, a ubiquitin promoter, at least one cloning site, a first polyadenylation sequence, a first origin of replication, at least one SV40 promoter that includes an SV40 origin, a first antibiotic resistance marker, a second polyadenylation sequence, a third polyadenylation sequence, a second origin of replication, and a second antibiotic resistance marker.

### BACKGROUND OF THE INVENTION

1. Field of the Invention

The present invention relates to vectors, methods of transfecting cells with the vectors, transfected cells, and antibiotic resistance cassettes. For instance, the present invention may be used to clone nucleotide sequences and express the peptides or proteins encoded by the nucleotide sequences.

2. Background Art

Many peptides of interest and/or potential industrial or medical importance, including hormones, enzymes and viral capsid antigens, are difficult to isolate in sufficient quantities from their natural sources. One approach to this problem has been to utilize methods of recombinant DNA technology to excise gene sequences coding for peptide compounds of interest and recombine them into self-replicating vectors. When placed in appropriate host cells, the recombined vectors can direct synthesis of desired peptides in amounts significantly greater than can be isolated from nature.

The success of processes for the production of polypeptides by recombinant DNA methods is largely dependent on the vector chosen for cloning and expression. Ideally, the vector should combine a variety of features such as convenient restriction enzyme cleavage sites to facilitate nucleic acid insertion, elements to ensure high copy number and efficient transcription and translation, a regulatory mechanism to control expression of the inserted sequence, and a marker gene to detect the presence of the vector in its host.

2.1. Recombinant DNA Technology and Gene Expression

Recombinant DNA technology involves insertion of specific DNA sequences into a DNA vector (vehicle) to form a recombinant DNA molecule capable of replication in a host cell. Generally, the inserted DNA sequence is foreign to the recipient DNA vehicle, i.e., the inserted DNA sequence and the DNA vector are derived from organisms that do not exchange genetic information in nature, or the inserted DNA sequence may be wholly or partially synthetically made.

Several general methods have been developed that enable construction of recombinant DNA molecules. For example, U.S. Pat. No. 4,237,224 to COHEN et al. describes production of such recombinant plasmids using restriction enzymes and a method known as ligation. These recombinant plasmids are then introduced, by means of transformation, and replicated in unicellular organisms. Another method for introducing recombinant DNA molecules into unicellular organisms is transduction or transfection that utilizes bacteriophage vectors and an in vitro packaging system (see U.S. Pat. No. 4,304,863 to COLLINS et al.).

Regardless of the method used for construction, the recombinant DNA molecule must be able to survive and replicate in the host cell. The recombinant DNA molecule should also have a marker function that allows the selection of host cells so transformed (or transduced) by the recombinant DNA molecule. In addition, if all of the proper replication, transcription, and translation signals are correctly arranged on the plasmid, the foreign gene will be properly expressed in the transformed cells and their progeny.

The processes of transcription and translation represent two levels of control of gene expression. Transcription of DNA is dependent on the presence of a promoter, a DNA sequence that directs the binding of RNA polymerase and thereby promotes transcription of a gene or a group of linked genes (operon). Promoters vary in their “strength”, i.e., their ability to promote transcription. For the purpose of molecular cloning, it is desirable to use strong promoters to obtain a high level of transcription and, hence, expression of the gene. Depending on the host cell system utilized, any one of a number of suitable promoters may be used. For instance, when cloning in an E. coli host cell system, any of the promoters isolated from E. coli its bacteriophages or plasmids may be used. More specifically, the PR and PL promoters of coliphage λ direct high levels of transcription of adjacent DNA segments. In addition, the recA and lac promoters from E. coli provide high levels of gene transcription of adjacent fragments. Furthermore, other E. coli promoters or synthetic DNA sequences may be used to provide the signal for transcription of the inserted gene.

Any of the methods previously described (e.g., U.S. Pat. No. 4,237,224) for the insertion of DNA fragments into a vector may be used to ligate a promoter segment and any control elements into specific sites within the vector.

Similarly, a gene of interest (or any portion thereof) can be inserted into an expression vector at a specific site in relation to the promoter and other control elements so that the gene sequence can be expressed correctly on the plasmid. The resultant recombinant DNA molecule is then introduced into appropriate host cells by transformation, transduction, or transfection (depending upon the vector/host cell system). Transformants may be selected on the basis of the expression of an appropriate marker gene included, and known to be able to be expressed, on the vector in an appropriate host cell, such as ampicillin-resistance or tetracycline-resistance in E. coli or thymidine kinase activity in eucaryotic host cell systems. Expression of such marker proteins indicates that the recombinant DNA molecule entered the cell and is intact.

2.2. Plasmid Cloning and Expression Vehicles

Numerous investigators have applied recombinant DNA technology in recent years to the construction of maximally expressing plasmids and other nucleic acid cloning vectors. That is, the construction of vehicles capable of: (a) replicating in a host cell at high copy number, and (b) directing high levels of transcription and/or translation has been the objective of studies ultimately aimed at overproducing (poly)peptide products of gene sequences inserted into the expression vehicle using restriction endonuclease cleavage and ligation.

Examples of commercially available plasmids include pUB6N5-His A, B, and C, available from Invitrogen, Carlsbad, Calif.

In view of the limitations associated with current plasmid cloning and expression vectors, there remains a need for suitable plasmids capable of autonomous replication in host microorganisms and useful as vectors for the cloning and expression of recombined or inserted nucleic acid sequences.

### SUMMARY OF THE INVENTION

Accordingly, the present invention provides a plasmid cloning and expression vector capable of replication in a variety of host cells. Other features and advantages of the present invention will be set forth in the description of invention that follows, and in part will be apparent from the description or may be learned by practice of the invention. The invention will be realized and attained by the compositions and methods particularly pointed out in the written description and claims.

A first aspect of the present invention is directed to a vector that includes, from upstream to downstream, a first promoter, at least one cloning site, a rat Kv2.1 polyadenylation sequence, and an origin of replication. The invention is also directed to a method that includes transfecting cells with this vector to form transfected cells.

Another aspect of the invention is directed to a vector that includes, from upstream to downstream, a first promoter, an encoding nucleotide sequence that encodes one of hHNa, HKvLQT1, hminK, hKv1.5, hERG, and rKv4.3, an origin of replication, and a neomycin resistance cassette comprising a neomycin resistance gene, and an SV40 promoter that includes an SV40 origin. The invention is also directed to a method that includes transfecting cells with this vector to form transfected cells.

Still another aspect of the invention is directed to a vector that includes, from upstream to downstream, a ubiquitin promoter, at least one cloning site, a first polyadenylation sequence, a first origin of replication, at least one SV40 promoter that includes an SV40 origin, a first antibiotic resistance marker, a second polyadenylation sequence, a third polyadenylation sequence, a second origin of replication, and a second antibiotic resistance marker.

Yet another aspect of the invention is directed to a vector that includes, from upstream to downstream, a UbC promoter, multiple cloning sites, a Kv2.1 polyadenylation sequence, an f1 origin, a first SV40 promoter that includes a first SV40 origin, a neomycin resistance gene, a TK polyadenylation sequence, an SV40 polyadenylation sequence, a pMB1 origin, and an ampicillin resistance gene.

In other aspects, the present invention is directed to a vector that includes a nucleotide sequence that is at least 85% homologous to SEQ ID NO. 1 or SEQ ID NO. 2.

In yet another aspect, the present invention is directed to an antibiotic resistance cassette that includes, from upstream to downstream, a first SV40 promoter that includes a first SV40 origin, an antibiotic resistance gene, and a TK polyadenylation sequence.

In still another aspect, the present invention is directed to an antibiotic resistance cassette that includes, from upstream to downstream, a first SV40 promoter that includes a first SV40 origin, a second SV40 promoter that includes a second SV40 origin, and an antibiotic resistance gene.

In another aspect, the invention is directed to a vector that includes, from upstream to downstream, a TK polyadenylation sequence, and an SV40 polyadenylation sequence having a border that is within 500 nucleotides of a border of the TK polyadenylation sequence.

### BRIEF DESCRIPTION OF THE DRAWINGS

The present invention is further described in the description of invention that follows, in reference to the noted plurality of non-limiting drawings, wherein:

FIG. 1 is a schematic showing the plasmid pCTx.

FIG. 2 is a schematic showing the plasmid pCTlx.

FIG. 3 is a graph showing that a hERG/pCTx clone of the present invention is effective.

### DESCRIPTION OF THE INVENTION

The particulars shown herein are by way of example and for purposes of illustrative discussion of the various embodiments of the present invention only. In this regard, no attempt is made to show details of the invention in more detail than is necessary for a fundamental understanding of the invention, the description taken with the drawings making apparent to those skilled in the art how the several forms of the invention may be embodied in practice.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Before further discussion, a definition of the following term will aid in the understanding of the present invention. “Transfection” is the transfer of a nucleotide sequence into a cell.

As an overview, the present invention includes vectors useful for cloning and expressing nucleic acid sequences, methods of transfecting cells with these vectors, and transfected cells containing these vectors. The present invention also includes components of such vectors, including antibiotic resistance cassettes. The vectors of the present invention generally comprise a regulatory sequence, such as a promoter or an origin of replication, and at least one cloning site. The antibiotic resistance cassette of the present invention generally comprises a regulatory sequence and an antibiotic resistance gene, such as a gene conferring resistance to neomycin or ampicillin.

Cloning Sites

The vectors of the present invention include at least one cloning site that allows insertion of a nucleic acid sequence of interest, such as a gene of interest. In one embodiment, the cloning site(s) comprise at least one restriction site, i.e., a site where the vector may be selectively cleaved by a particular enzyme. Such sites are known to those skilled in the art. The restriction site may be a unique restriction site, i.e., a restriction site not found elsewhere in the vector or nucleic acid sequence of interest. The cloning site of the inventive vectors may comprise a plurality of unique restriction sites to permit insertion of a wide variety of nucleic acid sequences. Illustrative examples of restriction sites include, but are not limited to, the following: HindIII site, BamHI site, Asp718I site, Kpn I site, Bst I site, EcoRI site, EcoRV site, PstI site, Eco32I site, XhoI site, Sfr274I site, XbaI site, FauNDI site, NdeI site, and PmeI site.

Sequences of Interest

The at least one cloning site of the present invention may include one or more sequences (“genes of interest”) for cloning and/or expressing one or more products of interest. Such sequences are commercially available, for example, green fluorescent protein (G.P.) is available from Clontech, Palo Alto, Calif., and luciferase is available from Promega, Madison, Wis., or may be obtained according to methods and techniques known to those skilled in the art.

For example, nucleic acid sequences from a selected source can be isolated by standard procedures, which typically include successive phenol and phenol/chloroform extractions followed by ethanol precipitation. After precipitation, the polynucleotides can be treated with a restriction endonuclease that cleaves the nucleic acid molecules into fragments. Fragments of the selected size can be separated by a number of techniques, including agarose or polyacrylamide gel electrophoresis or pulse field gel electrophoresis (CARE et al., Nuc. Acid Res., 12:5647-5664 (1984); CHU et al., Science, 234:1582 (1986); SMITH et al., Methods in Enzymology, 151:461 (1987)), to provide an appropriate size starting material for cloning.

Another suitable method of obtaining the nucleotide components of the expression vectors or constructs is PCR (polymerase chain reaction). General procedures for PCR are taught in MacPHERSON et al., PCR: A Practical Approach (1991). PCR conditions for each application reaction may be empirically determined. A number of parameters influence the success of a reaction. Among these parameters are annealing temperature and time, extension time, Mg⁺² and ATP concentration, pH, and the relative concentration of primers, templates, and deoxyribonucleotides. After amplification, the resulting fragments can be detected by agarose gel electrophoresis followed by visualization with ethidium bromide staining and ultraviolet illumination.

Yet another suitable method for obtaining polynucleotides is by enzymatic digestion. For example, nucleotide sequences may be generated by digestion of appropriate vectors with suitable recognition restriction enzymes. Restriction cleaved fragments may be blunt ended by treating with the large fragment of E. coli DNA polymerase I (Klenow) in the presence of the four deoxynucleotide triphosphates (dNTPs) using standard techniques.

The vectors described herein are useful for cloning and/or expressing any nucleic acid sequence of interest. The sequences of interest may be homologous or heterologous DNA whose expression at an elevated level is desired. Accordingly, the sequence of interest employed in this invention may encode a functional polypeptide, such as an amino acid sequence that possesses a biological activity, or an amino acid sequence that is a precursor of a protein having a biological activity, or a regulatory element, such as a promoter or repressor. The sequence of interest will generally encode a native or recombinant protein, although the expression of other polypeptides, such as epitopes or other immunologically active polypeptides, is also contemplated. Illustrative examples of proteins that can be expressed using the vectors and methods of this invention include, but are not limited to, hormones; cytokines, such as growth factors; enzymes; receptors; oncogenes; polypeptide vaccines; viral proteins; and structural and secretory proteins. For instance, the sequence of interest may encode a membrane ion-channel protein, such as hERG (human ether-a-go-go), hHNa, HKvLQT1, hmink, hKv1.5, hERG, and rKv4.3. The nucleic acid employed in the constructs of the invention may be cDNA sequences or sequences that retain intronic regions.

Regulatory Sequences

In addition to the cloning site(s) and/or sequence(s) of interest, the vectors of the present invention further comprise at least one regulatory element. The regulatory elements direct cloning and/or expression of the sequence(s) of interest. Regulatory elements, and their sequences, are known and available to those skilled in the art. Examples of regulatory elements include, but are not limited to, promoters, origins of replication, and other homologous or heterologous regulatory elements (e.g., affecting transcription and/or translation, as well as post-translational events and modifications).

Expression of the sequence of interest may be constitutive, or may be controllable, for example, by use of one or more regulatory elements. Regulatory elements may be selected, in part, based on their compatibility with and utility in the intended host cell. Illustrative examples of such regulatory elements include, but are not limited to, transcription promoters, transcription enhancer elements, transcription termination signals, polyadenylation sequences (located 3′ to the translation stop codon), sequences for optimization of initiation of translation (located 5′ to the coding sequence), translation termination sequences, secretion signal sequences, and sequences that direct post-translational modification (e.g., glycosylation sites). Transcription promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters.

The vectors of the present invention include at least one promoter upstream of the cloning site(s) such that the promoter initiates productive translation of the sequence of interest. For example, the promoter employed in the inventive vector may be heterologous to the gene of interest (for example, SV40 promoter and neomycin resistance gene). Alternatively, the promoter may be homologous to the peptide coding sequences (for example, human glucose-6-phosphate dehydrogenase under the control of its own transcription promoter sequences).

Promoters for use in eukaryotic host cells are known to those skilled in the art. Illustrative examples of such promoters include, but are not limited to, promoters from Simian Virus 40 (SV40), Mouse Mammary Tumor Virus (MMTV) promoter, Human Immunodeficiency Virus (HIV) promoters, such as the HIV Long Terminal Repeat (LTR) promoter, Moloney virus promoters, ALV promoters, cytomegalovirus (CMV) promoters, such as the CMV immediate early promoter, Epstein Barr Virus (EBV) promoter, Raus Sarcoma Virus (RSV) promoter, as well as promoters from human genes such as human actin, human myosin, human hemoglobin, human muscle creatine, and human metalothionein. Still other examples of suitable promoters include the CAG promoter (a hybrid promoter comprising a CMV enhancer, a chicken β-actin promoter, and a rabbit β-globin splicing acceptor, and poly(A) sequence).

The promoter may be a ubiquitin promoter, such as a human ubiquitin promoter, such as a human ubiquitin C (UbC) promoter. The human UbC promoter permits overexpression of recombinant protein in a broad range of mammalian cell types. HERSKO et al., Ann. Rev. Biochem., 51:335-364 (1982); WULFF et al., FEBS Lett., 261:101-105 (1990); and SCHORPP et al., Nuc. Acids Res., 24:1787-1788 (1996).

The promoter upstream of the cloning sites may be an inducible promoter, such as an inducible promoter that is normally inactive in the host cell and strongly active in the presence of inducing agent(s). Inducible promoters include, but are not limited to, lac, trp, and tac from E. Coli, P_{R} and P_{L} promoters from bacteriophage λ. Illustrative examples include E. Coli lac T and lac Z promoters, T3 and T7 promoters, and gpt promoter.

The inventive vectors preferably further comprise at least one origin of replication useful for propagation in the desired host cell. Origins of replication are known and available to those skilled in the art, and include both viral and animal origins. For instance, the origin may be an f1 origin that allows rescue of single-stranded DNA in E. coli. The origin is typically 5′ (upstream) of the cloning site(s).

The origin may be a conditional origin of replication, such as oriV (GenBank No. L13843), pBR1, mb1, or RSF1010, but could be any origin that functions in the host cell and is normally inactive until exposed to replication inducing agent(s). Replication may be induced by a single agent, such as a protein (although multiagent replication systems are known and available to those skilled in the art). If the inducing agent is encoded by a polynucleotide, that sequence can be provided in an expression cassette under the control of an inducible promoter, which may be the same as or different from the other promoter(s) present in the inventive vectors. Such an expression cassette may be included in the inventive vectors or may be provided in the host cell genome or on a plasmid.

The vectors of the present invention may also include other expression regulatory elements, such as one or more polyadenylation sequences (e.g., SV40, poly(A), LTR poly(A), rabbit β-globin poly(A), or bovine growth hormone (BGH) polyadenylation sequence). The polyadenylation sequence is typically 3′ or downstream of the cloning site(s).

In one aspect, the polyadenylation sequence is a rat Kv2.1 polyadenylation sequence. This polyadenylation sequence provides efficient transcription termination and polyadenylation of mRNA.

Vectors may also include an enhancer sequence, such as those from human actin, human myosin, human hemoglobin, human muscle creatine, and viral enhancers, such as those from CMV, RSV, and EBV.

Selectable Markers

The vectors of the present invention generally include at least one selectable marker. Any suitable sequence encoding for a selectable marker can be used as a marker. The selectable marker genes may be obtained from readily available sources.

A number of selection systems may be used, including but not limited to the herpes simplex virus thymidine kinase (WIGLER et al., Cell, 11:223 (1977)), hypoxanthine-guanine phosphoribosyltransferase (SZYBALSKA et al., Proc. Natl. Acad. Sci. USA, 48:202 (1992)), and adenine phosphoribosyltransferase (LOWY et al., Cell, 22:817 (1980)) genes can be employed in tk-, hgprt- or aprt-cells, respectively. Also, antimetabolite resistance can be used as the basis of selection for the following genes: dhfr, which confers resistance to methotrexate (WIGLER et al., Natl. Acad. Sci. USA, 77:357 (1980); O'HARE et al., Proc. Natl. Acad. Sci. USA, 78:1527 (1981)); gpt, which confers resistance to mycophenolic acid (MULLIGAN et al., Proc. Natl. Acad. Sci. USA, 78:2072 (1981)); neo, which confers resistance to the aminoglycoside G418 (Clinical Pharmacy, 12:488-505; WU et al., Biotherapy, 3:87-95 (1991); TOLSTOSHEV, Ann. Rev. Pharmacol. Toxicol., 32:573-596 (1993); MULLIGAN, Science, 260:926-932 (1993); and MORGAN et al., Ann. Rev. Biochem., 62:191-217 (1993); TIBTECH 11(5):155-215 (May 1993)); and hygro, which confers resistance to hygromycin (SANTERRE et al., Gene, 30:147 (1984)). Methods commonly known in the art of recombinant DNA technology may be applied to select the desired recombinant clone, and such methods are described, for example, in AUSUBEL et al., Current Protocols in Molecular Biology (1993); KRIEGLER, Gene Transfer and Expression, A Laboratory Manual (1990); and in Chapters 12 and 13, DRACOPOLI et al., Current Protocols in Human Genetics (1994); COLBERRE-GARAPIN et al., J. Mol. Biol., 150:1 (1981).

Examples of antibiotic resistance genes include those conferring resistance to at least one of neomycin (neo), ampicillin, blasticidin, kanamycin (kan), methotrexate, tetracycline, spectinomycin, erythromycin, chloramphenicol, phleomycin, Tn917, gentamycin, and bleomycin. An example of the neomycin resistance gene is the neomycin resistance gene of transposon Tn5 that encodes for neomycin phosphotransferase 11, which confers resistance to various antibiotics, including G418 and kanamycin. The optimum amount of substrate (e.g., G418) needed for selection can be individually determined for each cell line. Other similar selectable markers include, but are not limited to, the following.

Temperature-sensitive selectable markers can also be employed. For example, temperature-sensitive neo will be nearly wild type in function at non-stringent temperature and have low activity at stringent temperature. After electroporation, insertion can be performed using G418 at non-stringent temperature. After colonies begin to grow, stringent temperature can be used to kill off colonies carrying low expression insertions.

It will be understood that other selectable markers, which permit isolation of stable transfectants, can be employed in this invention as markers. An example of another selectable marker is adenosine deaminase (ADA). A medium supplemented with thymidine, 9-β-D-xylofuranosyl adenine (Xyl-A), and 2′-deoxycoformycin (dCF) is employed. Xyl-A can be converted to Xyl-ATP and incorporated into nucleic acids, resulting in cell death. Xyl-A is detoxified to its inosine derivative by ADA. dCF is a transition state analogue inhibitor of ADA, and is needed to inactivate ADA endogenous to the parental cell type. As the level of endogenous ADA varies with cell type, the appropriate concentration of dCP for selection will vary as well. KAUFMAN et al., PNAS USA, 83:3136-3140 (1986). ADA-deficient CHO cells are also available as host cells.

Another suitable selectable marker is thymidine kinase (TK). In forward selection (TK⁻ to TK⁺), complete medium is supplemented with hypoxanthine, aminopterin, thymidine, and glycine (HAT medium). In reverse selection (TK⁺ to TK⁻), complete medium is supplemented with 5-bromodeoxyuridine (BrdU). Under normal growth conditions, cells do not need thymidine kinase, because the usual means for synthesizing dTDP is through dCDP. Addition of BrdU to the medium will kill TK⁺ cells, as BrdU is phosphorylated by TK and then incorporated into DNA. Selection of TK⁺ cells in HAT medium is primarily due to the presence of aminopterin, which blocks the formation of dTDP from dCDP. Cells, therefore, need to synthesize dTDP from thymidine, a pathway that requires TK. Thymidine kinase is widely used in mammalian cell culture because both forward and reverse selection conditions exist. Like ADA, most mammalian cell lines express TK, removing the possibility of using the marker in those lines unless BrdU is used to select a TK⁻ mutant. See LITTLEFIELD et al., Science, 145:709-710 (1964).

An example of another suitable dominant selectable marker for use in the invention is xanthine-guanine phosphoribosyltransferase (XGPRT, gpt). Medium containing dialyzed fetal calf serum and xanthine, hypoxanthine, thymidine, aminopterin, mycophenolic acid, and L-glutamine can be employed. Aminopterin and mycophenolic acid both block the de novo pathway for synthesis of GMP. Expression of XGPRT allows cells to produce GMP from xanthine, allowing growth on medium that contains xanthine, but not guanine. XGPRT is a bacterial enzyme that does not have a mammalian homolog, allowing XGPRT to function as a dominant selectable marker in mammalian cells. The amount of mycophenolic acid necessary for selection varies with cell type and can be determined by titration in the absence and presence of guanine. See MULLIGAN et al., PNAS USA, 78:2072-2076 (1981).

The selectable marker hygromycin-B-phosphotransferase (HPH) can also be employed. Complete medium is supplemented with hygromycin-B. Hygromycin-B is an aminocyclitol that inhibits protein synthesis by disrupting translocation and promoting mistranslation. The HPH gene has been used in mammalian systems, and vectors that efficiently express the gene are available. See GRITZ et al., Gene, 25:179-188 (1983); and PALMER et al., PNAS USA, 84:1055-1059 (1987).

Another useful marker is chloramphenicol resistance. Resistance is mediated by chloramphenicol acetyltransferase (CAT), which inactivates chloramphenicol by converting it into mono- and bi-acetylated derivatives. These derivatives can be detected by thin layer chromatography. This enzyme is expressed in mammalian cells and is easily detected because it does not naturally occur in mammalian cells. The gene can be obtained from a derivative of PBR322 carrying transposon Tn9 by cleavage with suitable enzymes.

GOSSEN et al., Science, 268:1766-1769 (1995), describes fusion of a tetracycline resistance gene repressor to a viral transcription activation domain in order to induce rapid, greatly amplified gene expression in the presence of tetracycline. It is a modification of a preexisting system in which low levels of tetracycline prevented gene expression. The gene that codes for the tetracycline resistance gene repressor was mutagenized, and a mutant fusion protein was created that depended on tetracycline for activation. The construct can provide an on/off switch for high expression of a gene.

Another suitable marker is adeninephosphoribosyl transferase (APRT). The enzyme APRT, another enzyme of the purine salvage pathway, catalyzes the conversion of adenine to AMP. APRT positive cells can be selectable in a medium containing, for example, the glutamine analogue azaserine, which prevents de novo synthesis of purines. APRT-negative cells cannot be grown in a medium containing azaserine and adenine, and can be selected by treatment with 2,6-diaminopurine. This compound is toxic for normal cells, but APRT-negative cells survive because they do not incorporate it.

In one aspect of the invention, the vector may be expressed in either bacterial or mammalian cells. A first selectable marker allows selection of transfected bacterial cells from untransfected cells. A second selectable marker allows selection of transfected mammalian cells from untransfected cells. For instance, the first selectable maker may encode a gene that confers resistance to a first antibiotic, and the second selectable marker may confer resistance to a second antibiotic. As an example, the first selectable marker may comprise a neomycin (neo) resistance gene that allows selection of transfected mammalian cells, and the second selectable marker may comprise an ampicillin resistance gene that allows selection of transfected bacterial cells.

The expression of the selectable marker coding sequences can be placed under the control of, for example, promoter sequences derived from CMV, RSV, SV40, or the like, and may include other expression control elements as well (e.g., sequences affecting transcription, translation, or post-translation modifications).

In one aspect, the selectable marker comprises a neomycin resistance gene that is under the control of an SV40 promoter that includes an SV40 origin.

In another aspect, a selectable marker is connected to a pMB1 origin. The pMB1 origin gives high copy number replication and growth in E. coli.

The selectable markers may be contained within a cassette comprising an upstream promoter, such as CMV, SV40, RSV, and HSV-TK promoters, and a downstream polyadenylation sequence, such as BGH polyA, TK polyA, or SV40 polyA.

In one aspect, a selectable marker is connected to a TK polyadenylation sequence that provides efficient transcription termination and polyadenylation of mRNA.

In one aspect, the present invention is directed to an antibiotic resistance cassette. The antibiotic resistance cassette may confer resistance to antibiotics discussed above, such as neomycin, blasticidin, and ampicillin. The antibiotic resistance cassette may comprise a promoter, an origin, and an antibiotic resistance gene. The promoter may be an SV40 promoter that includes an SV40 origin. The antibiotic resistance cassette may comprise a plurality of promoters, such as two SV40 promoters. The antibiotic resistance cassette may also comprise a polyadenylation sequence, such as a TK polyadenylation sequence, such as a TK polyadenylation sequence from pCR3 (Invitrogen, Carlsbad, Calif.).

Backbone Vectors

The above-described components can be incorporated into a number of suitable backbone vectors to facilitate manipulation of the expression vectors and constructs. For example, incorporation of the components into a vector containing means that allow replication in a microorganism greatly facilitates propagation and isolation of the constructs (i.e., creating shuttle vectors). A variety of such backbone vectors are available for appropriate host systems, discussed below. Exemplary backbone vectors include, but are not limited to, the following: pCMV6a and pUC19.

Examples of the vectors of the present invention with multiple cloning sites, but without gene(s) of interest, include pCTx (SEQ ID NO. 1; Example 1) and pCTlx (SEQ ID NO. 2; Example 2). Additional examples include vectors that are at least 85%, 90%, 93%, 95%, 98%, or 99% homologous to SEQ ID NO. 1 or SEQ ID NO. 2.

Other examples of vectors of the present invention include those having, from 5′ to 3′, an inducible promoter, such as a ubiquitin promoter, at least one cloning site, a first polyadenylation sequence, a first origin of replication, at least one promoter, such as an SV40 promoter, at least one origin, such as an SV40 origin, a first selectable marker, such as an antibiotic resistance marker, a second polyadenylation sequence, a third polyadenylation sequence, a second origin of replication, and a second selectable marker, such as an antibiotic resistance marker.

Still another example of a vector with at least one cloning site, but without a gene of interest, is one having, from upstream to downstream, a UbC promoter, multiple cloning sites, a Kv2.1 polyadenylation sequence, an f1 origin, a first SV40 promoter that includes a first SV40 origin, a neomycin resistance gene, a TK polyadenylation sequence, an SV40 polyadenylation sequence, a pMB1 origin, and an ampicillin resistance gene.

An example of the vector with a gene of interest includes one having a promoter, a nucleotide sequence encoding hERG, an origin of replication, and a neomycin resistance cassette comprising a neomycin resistance gene, and an SV40 promoter that includes an SV40 origin.

Synthesis

The vectors of the present invention can be produced following the teachings of the present specification in view of techniques known in the art. For example, polynucleotides may be inserted into cloning sites of suitable vectors, for example, plasmids, using methods known in the art. Insert and vector DNA may be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers may contain nucleic acid sequences that correspond to a particular restriction site in the vector DNA. Other means are known in the art. A variety of sources can be used for the component polynucleotides.

These methods are known in the art and are described, for example, in MILLER, Experiments in Molecular Genetics (1972); MILLER, A Short Course in Bacterial Genetics (1992); SINGER et al., Genes & Genomes (1991); SAMBROOK et al., Molecular Cloning: A Laboratory Manual, 2d ed., (1989); KAUFMAN, Handbook of Molecular and Cellular Methods in Biology and Medicine (1995); GLICK et al., Methods in Plant Molecular Biology and Biotechnology (1993); and SMITH-KEARY, Molecular Genetics of Escherichia coli (1989).

Replication and Expression

Expression and replication of the vectors of the present invention may occur in the same or different hosts. These host systems include, but are not limited to, the following: baculovirus (REILLY et al., Baculovirus Expression Vectors: A Laboratory Manual (1992); BEAMES et al., Biotechniques, 11:378 (1991); Pharmingen; Clontech, Palo Alto, Calif.); pAcCl3, a shuttle vector for use in the Baculovirus expression system derived from pAcC12, MUNEMITSU et al., Mol Cell Biol., 10(11):5977-5982 (1990)), bacteria (pBR322; AUSUBEL et al., Current Protocols in Molecular Biology; Clontech; Promega, Madison, Wis.; Life Technologies, Gaithersburg, Md.), yeast (U.S. Pat. No. RE 35,749 to ROSENBERG et al.; U.S. Pat. No. 5,629,203 to SHUSTER; ROMANOS et al., Yeast, 8(6):423-488 (1992); GOEDDEL, Methods in Enzymology, 185 (1990); GUTHRIE et al., Methods in Enzymology, 194 (1991)), mammalian cells (Clontech; Promega, Madison, Wis.; Life Technologies, Gaithersburg, Md.; e.g., Chinese hamster ovary (CHO) cell lines (HAYNES et al., Nuc. Acid. Res., 11:687-706 (1983); LAU et al., Mol. Cell. Biol., 4:1469-1475 (1984))), and plant cells (plant cloning vectors, Clontech Laboratories, Inc., Palo Alto, Calif., and Pharmacia LKB Biotechnology, Inc., Pistcataway, N.J.; HOOD et al., J. Bacteriol., 168:1291-1301 (1986); NAGEL et al., FEMS Microbiol. Lett., 67:325 (1990); AN et al., “Binary Vectors,” Plant Molecular Biology Manual, A3:1-19 (1988); MIKI et al., Plant DNA Infectious Agents, 249-265 (1987); JONES et al., Plant Molecular Biology: Essential Techniques (1997); MIGLANI, Gurbachan Dictionary of Plant Genetics and Molecular Biology (1998); HENRY, Practical Applications of Plant Molecular Biology (1997)).

The hosts, e.g., host cells, may be grown in the presence of the appropriate substrate for the selectable marker, for example, ampicillin or G418 if the selectable marker encodes neomycin. Cells that survive selection in high concentration of the antibiotic have integrated the resistance gene at a high expression locus.

After replication, the vectors and constructs described herein may be introduced into a different host by a variety of methods. For instance, mammalian cells may be transfected or infected with a vector. Transfection can be carried out by known techniques, such as calcium phosphate transfection, DEAE-dextran mediated transfection, electroporation, liposome mediated transfection, or microinjection (AUSUBEL et al., supra). Transfection can be employed with DNA fragments that are unable to replicate, or with DNA that is not readily packaged in viral vectors, or where infection of the mammalian cells with viral DNA is to be avoided.

Appropriate transformation/transfection conditions can be determined by those skilled in the art in view of the teachings herein.

The cells (e.g., host cells) for expression include all mammalian cells, cell lines, and cell cultures. The cells can be derived from mammals, such as mice, rats, or other rodents, or from primates, such as humans or monkeys. Mammalian germ cells or somatic cells can be employed for this purpose. Primary cell cultures or immortalized cells can be employed in carrying out the techniques of this invention.

The mammalian cells are typically grown in cell culture for transformation by the DNA. The cells can be fixed to a solid surface or grown in suspension in appropriate nutrient media.

Expression of the gene of interest in the mammalian cells can be stable or transient. Even transient expression at a higher than normal level is useful for functional studies in the cells or for the production and recovery of proteins of interest.

It is preferred that permanent (i.e., stable) transformation occurs. This is accompanied by integration of the transforming DNA into the cellular genome by recombination. Insertional transformation, which results in the high expression locus being tagged, usually takes place by non-homologous recombination of the DNA construct containing the tag into a random genomic position, although it will be understood that homologous recombination can occur.

No attempt has been made to determine whether the selectable markers integrate in a single high expression locus in chromosomal DNA or whether there are multiple sites of integration to form multiple high expression loci in a given cell. In any event, the mammalian cells of this invention contain at least one high expression locus.

The transformed cells obtained by the method of this invention can be employed for the preparation of continuous cell lines in which the cells are essentially immortal, or for the preparation of established cell lines that have the potential to be subcultured in vitro. Continuous cell lines and established cell lines can be obtained from a variety of organisms and organs, such as rodent embryos; primate kidneys; rodent and human tumors; and fibroblast, epithelial, or lymphoid cells. Cells exhibiting the highest levels of expression can be cloned, if desired.

Examples of established cell lines that can be transformed by the techniques of this invention include BHK, VERO, HT1080, 293, RD, COS-7, HEK, e.g., HEK 293, HeLa, CV-1, CHO, 3T3, L, and TC7. All of these cells are sensitive to aminoglycoside antibiotics, such as G418, and are capable of harboring kanamycin or neomycin resistance genes for expression therein.

The present invention will be further illustrated by way of the following Examples. These examples are non-limiting and do not restrict the scope of the invention.

### EXAMPLE 1

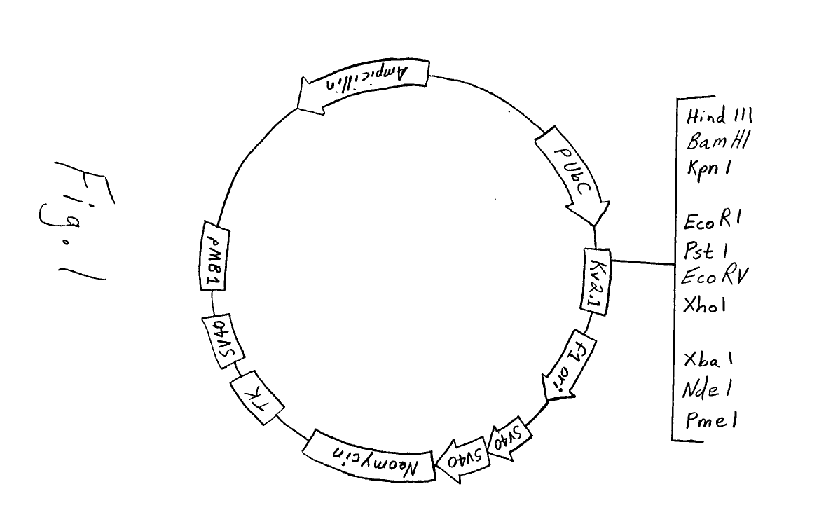

A vector (pCTx) having a sequence corresponding to SEQ ID NO. 1 was made by conventional techniques. The vector (pCTx) is shown schematically in FIG. 1.

### EXAMPLE 2

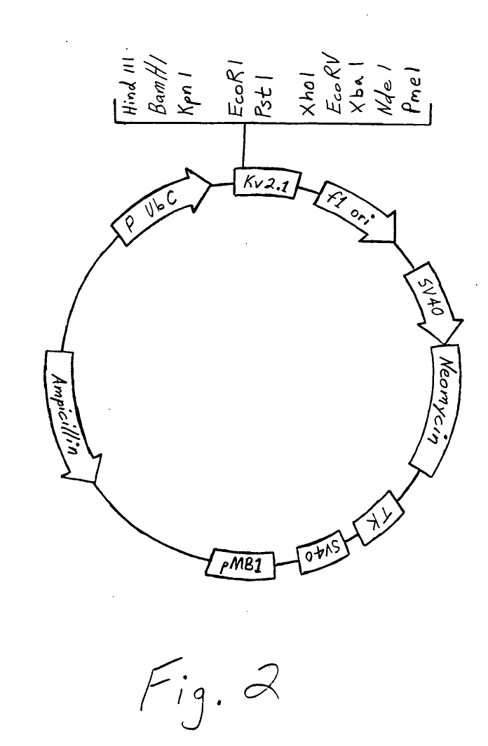

A vector (pCTlx) having a sequence corresponding to SEQ ID NO. 2 was made by conventional techniques. The vector (pCTIx) is shown schematically in FIG. 2.

### EXAMPLE 3

Sequences encoding hERG (human ether-a-go-go) were inserted into pCTx using standard procedures. The resulting plasmids were transfected into E. coli. The E. coli were then cultured to form colonies. The colonies of the transfected E. coli were then screened for resistance to ampicillin to determine which colonies include recombinant plasmids.

The recombinant plasmids were removed from the E. coli and then transfected into HEK 293 cells. The transfected HEK 293 cells were cultured. The HEK 293 cells were screened for resistance to neomycin to determine which cells included the recombinant plasmid.

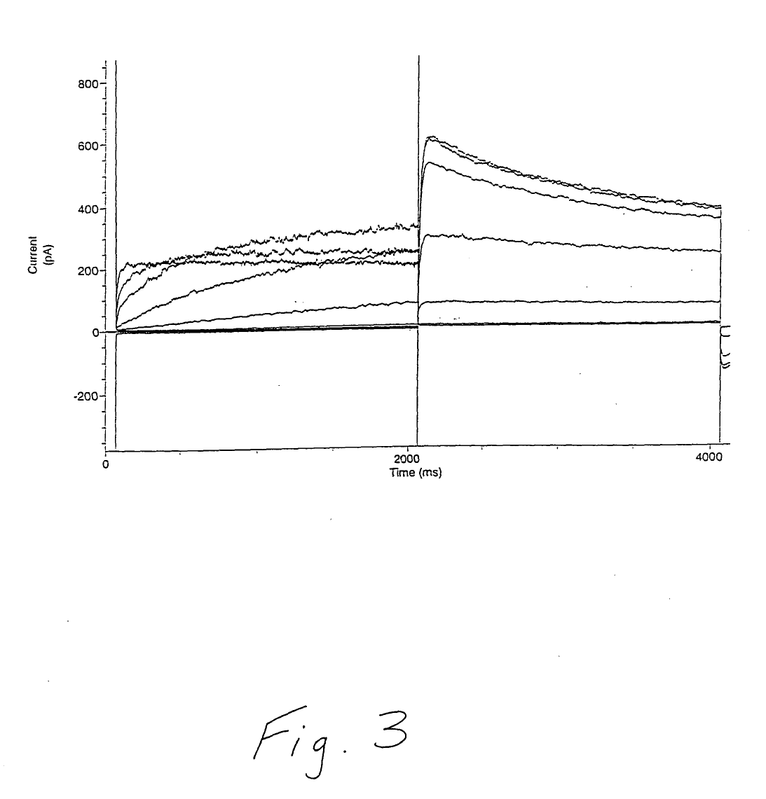

To show that the transfection was successful, a voltage was applied to the transfected HEK 293 cells. The holding potential was −80 mV, and 2 second pulses from −100 to +60 mV with 20 mV increments were applied every 10 seconds. Each voltage step was followed by a 2 second hyperpolarizing pulse to −50 mV, which resulted in an outward transient (tail) current. The pipette solution was 140 mM Kaspartate, 5 mM MgCl₂, 10 mM HEPES, 10 mM EGTA, and 2 mM K₂ATP, with a pH of 7.2. The bath solution was 137 mM NaCl, 4.0 mM KCl, 1 mM MgCl₂, 1.8 CaCl₂, 10 HEPES, and 10 mM glucose, with a pH of 7.4. The results are shown in FIG. 3. The presence of a peak in FIG. 3 indicates that the HEK 293 cells were stably transfected with hERG channels.

The foregoing embodiments and advantages are merely exemplary and are not to be construed as limiting the present invention. The description of the present invention is intended to be illustrative, and not to limit the scope of the claims. Many alternatives, modifications, and variations will be apparent to those skilled in the art.

1. A vector, comprising from upstream to downstream:
a first promoter;
at least one cloning site;
a rat Kv2.1 polyadenylation sequence; and
an origin of replication.

2. The vector of claim 1, wherein the first promoter comprises a ubiquitin promoter.

3. The vector of claim 1, wherein the first promoter comprises a human ubiquitin promoter.

4. The vector of claim 1, wherein the at least one cloning site includes at least one restriction site selected from HindIII site, BamHI site, Asp718I site, Kpn I site, Bst I site, EcoRI site, EcoRV site, PstI site, Eco32I site, XhoI site, Sfr274I site, XbaI site, FauNDI site, and PmeI site.

5. The vector of claim 1, wherein the at least one cloning site includes an insert encoding a peptide.

6. The vector of claim 5, wherein the peptide comprises an ion-channel peptide.

7. The vector of claim 6, wherein the ion-channel peptide is selected from hERG, hHNa, HKvLQT1, hminK, hKv1.5, and rKv4.3.

8. The vector of claim 1, wherein the origin of replication is selected from f1, oriV, pBR1, pMB1, pUC1, and RSF1010 origins.

9. The vector of claim 1, further comprising a first selectable marker.

10. The vector of claim 9, wherein the first selectable marker comprises a first antibiotic resistance cassette.

11. The vector of claim 10, wherein the first antibiotic resistance cassette confers resistance to at least one of neomycin, blasticidin, ampicillin, kanamycin, methotrexate, tetracycline, spectinomycin, erythromycin, chloramphenicol, phleomycin, Tn917, gentamycin, and bleomycin.

12. The vector of claim 10, wherein the first antibiotic resistance cassette comprises a neomycin resistance cassette.

13. The vector of claim 10, wherein the first antibiotic resistance cassette comprises a blasticidin resistance cassette.

14. The vector of claim 10, wherein the first antibiotic resistance cassette comprises a promoter, an origin, and an antibiotic resistance gene.

15. The vector of claim 12, wherein the neomycin resistance cassette comprises a promoter and a neomycin resistance gene.

16. The vector of claim 15, wherein the promoter of the neomycin resistance cassette comprises an SV40 promoter that includes an SV40 origin.

17. The vector of claim 14, wherein the first antibiotic resistance cassette further comprises a polyadenylation sequence.

18. The vector of claim 17, wherein the polyadenylation sequence of the first antibiotic resistance cassette comprises a TK polyadenylation sequence.

19. The vector of claim 18, wherein the TK polyadenylation sequence comprises a nucleotide sequence corresponding to a nucleotide sequence from pCR3 or pCR3.1.

20. The vector of claim 15, wherein the neomycin resistance cassette further comprises a polyadenylation sequence.

21. The vector of claim 20, wherein the polyadenylation sequence of the neomycin resistance cassette comprises a TK polyadenylation sequence.

22. The vector of claim 21, wherein the TK polyadenylation sequence comprises a nucleotide sequence corresponding to a nucleotide sequence from pCR3 or pCR3.1.

23. The vector of claim 9, wherein the first selectable marker comprises at least two promoters and at least two origins.

24. The vector of claim 10, wherein the first antibiotic resistance cassette comprises at least two promoters and at least two origins.

25. The vector of claim 1, further comprising a polyadenylation sequence.

26. The vector of claim 26, wherein the polyadenylation sequence comprises an SV40 polyadenylation sequence.

27. The vector of claim 9, further comprising a second selectable marker.

28. The vector of claim 10, further comprising a second antibiotic resistance cassette.

29. The vector of claim 28, wherein the second antibiotic resistance cassette comprises an origin and an antibiotic resistance gene.

30. The vector of claim 29, wherein the origin of the second antibiotic resistance cassette is selected from f1, oriV, pBR1, pMB1, pUC1, and RSF1010 origins.

31. The vector of claim 30, wherein the antibiotic resistance gene of the second antibiotic resistance cassette comprises an ampicillin resistance gene.

32. The vector of claim 1, wherein the vector comprises a plasmid.

33. The vector of claim 1, wherein the vector comprises a nucleotide sequence corresponding to SEQ ID NO. 1.

34. The vector of claim 1, wherein the vector comprises a nucleotide sequence corresponding to SEQ ID NO. 2.

35. A transfected cell comprising the vector of claim 1.

36. The transfected cell of claim 35, wherein the cell is a prokaryotic cell.

37. The transfected cell of claim 35, wherein the cell is a eukaryotic cell.

38. The transfected cell of claim 35, wherein the cell is a mammalian cell.

39. The transfected cell of claim 38, wherein the mammalian cell is an HEK cell.

40. The transfected cell of claim 39, wherein the HEK cell is an HEK 293 cell.

41. A transfected cell comprising the vector of claim 2.

42. A transfected cell comprising the vector of claim 4.

43. A transfected cell comprising the vector of claim 5.

44. A transfected cell comprising the vector of claim 6.

45. A transfected cell comprising the vector of claim 7.

46. A transfected cell comprising the vector of claim 8.

47. A transfected cell comprising the vector of claim 9.

48. A transfected cell comprising the vector of claim 12.

49. A transfected cell comprising the vector of claim 32.

50. A transfected cell comprising the vector of claim 33.

51. A transfected cell comprising the vector of claim 34.
